(19) United States
(12) Patent Application Publication
(10) Pub. No.: US 2026/0265135 A1
(43) Pub. Date: Sep. 10, 2026

(54) IMPROVED RECYCLED CONCRETE AGGREGATES AND METHODS FOR PRODUCING THE SAME

(71) Applicant: Horizon Bioinnovation AB, Lund (SE)

(72) Inventor: Gashaw MAMO, Lund (SE)

(21) Appl. No.: 19/165,630

(22) PCT Filed: Mar. 15, 2025
(86) PCT No.: PCT/EP2024/057090

§ 371 Date: Sep. 16, 2025

(30) Foreign Application Priority Data
Mar. 17, 2024 (SE) 2350302-2

(51) Int. Cl. C04B 18/167; C04B 14/28; C04B 20/02; C04B 40/00; C12N 1/205; C12R 1/01

(52) U.S. Cl. CPC C04B 18/167; C04B 14/28; C04B 20/026; C04B 40/0046; C12N 1/205; C12R 2001/01

(57) ABSTRACT

Disclosed is a method of recycling reclaimed concrete by adding a composition comprising an alkaliphilic and psychrophilic microbe capable of depositing calcium carbonate to the reclaimed concrete during or after crushing or grinding. Also disclosed is a method of improving the properties of recycled concrete aggregates during storage at temperatures below 20° C. by adding a composition comprising at least one alkaliphilic and psychrophilic microbial culture capable of depositing calcium carbonate to the recycled concrete aggregate. Further provided is concrete aggregate characterized in comprising at least one culture comprising alkaliphilic and psychrophilic microbes that are capable of inducing calcium carbonate deposition.

### FIELD

The disclosure relates to recycled concrete aggregates and methods for preparation thereof. The disclosure also relates to recycled concrete aggregates containing mineralization microbes.

### INTRODUCTION

Concrete is one of the widest used building materials, with an annual worldwide use of 30 billion tons. Per capita, this is an increase of threefold compared with the use of concrete in 1980, and the demand is growing more steeply than for other building materials such as wood and steel.

Concrete, made by adding sand and gravel to cement, has a large carbon footprint, primarily from the production of cement. There is therefore an increased pressure on the cement industry to reduce its carbon footprint.

Recycling concrete represents another way of reducing the overall carbon footprint. Concrete waste resulting from the demolition of buildings represents about half of the total construction and demolition waste. By recycling concrete, natural resources can be conserved, transportation cost and material waste reduced, landfill space saved and pollution from transport to landfills and dumps reduced. Thereby, the carbon footprint of concrete production and use is reduced.

Concrete is recycled by crushing concrete rubble, removing trash, wood, paper and metals in the process. The coarse concrete is subsequently washed and reused-most commonly for road construction, but higher quality recycled concrete can be reused as a raw material for generating new concrete, such as for building construction.

Compared with natural concrete, recycled concrete has larger porosity, lower apparent density and lower compressional strength, leading to poor mechanical properties and durability. To overcome these deficiencies, various methods have been employed including removing mortar attached to the surface of concrete particles, modifying particle shape etc., using methods such as grinding, heat and pressure treatment and particle shaping. Alternative chemical methods are also employed such as cement slurry treatment, polymer impregnation and carbonization curing. These processes however lead to high energy consumption, increased production time, secondary pollution and increased equipment wear.

Certain microbes can form mineral deposits in the earth's biogeochemical circulation system. These include bacteria that produce carbonate (CO₃⁻²) which reacts with calcium (Ca⁺²) ions in soil to generate calcium carbonate (CaCO₃), also called calcite. The deposited CaCO₃ fills cracks and pores on the surface of soil and building material, resulting in mechanical reinforcement of the structure.

Since adherent mortar and microcracks are the primary reasons for poor mechanical properties of recycled concrete aggregates, the use of microbially induced calcium carbonate precipitation (also called MICP) technology represents a promising avenue for recycling concrete.

Bacteria used in MICP (most commonly Bacillus) must obviously be biologically active under the conditions of the concrete when recycled. Bacterial strains used to date have the deficit of being most active at mesophilic temperatures, typically around 30° C. This temperature is adequate in many parts of the world, but in colder climate the bacterial strains used to date in MICP are largely inactive, rendering the bacteria unsuitable for use in recycling concrete in such climates. Other factors influencing the mineralization process induced by the bacteria must also be considered, including the calcium source, pH, nucleation source and the medium (i.e. nutrients for growth).

### SUMMARY

The present invention provides recycled concrete aggregates with improved properties and methods for production thereof, with the goal of overcoming, eliminating or mitigating deficiencies of the prior art, for example the deficiencies described in the above.

In an aspect, the invention relates to a method of recycling reclaimed concrete comprising crushing or grinding reclaimed concrete to obtain concrete aggregates, wherein at least one composition comprising an alkaliphilic and psychrophilic microbial culture capable of depositing calcium carbonate is added to the reclaimed concrete during or after the crushing or grinding, thereby obtaining a recycled concrete aggregate comprising at least one alkaliphilic and psychrophilic microbial culture.

The at least one composition can be added in liquid or dry form, during or after the crushing or grinding.

Also provided is a method of improving properties of recycled concrete aggregates at temperatures below 20° C., the method comprising adding a composition comprising at least one alkaliphilic and psychrophilic microbial culture capable of depositing calcium carbonate on the recycled concrete aggregate.

Also provided is a method of strengthening concrete containing recycled concrete aggregate, the method comprising immersing concrete comprising a mixture of recycled concrete aggregate and fresh aggregate in a liquid composition comprising at least one alkaliphilic and psychrophilic microbial culture capable of depositing calcium carbonate in the concrete.

Further provided are microbial strains capable of inducing deposition of CaCO₃, wherein the microbial strains are active (i.e. able to grow) under alkaline conditions and a temperature below 20° C., preferably 5-15° C.

### BRIEF DESCRIPTION OF THE DRAWINGS

FIG. 1 shows a schematic view of concrete recycling.

### DESCRIPTION

The present invention provides methods of recycling concrete aggregates using microbial species that can form mineral deposits in concrete aggregates at low temperatures. By using certain psychrophilic microbes, surprising advantages in the recycling of concrete aggregates, as well as in the storage of such aggregates, have been discovered.

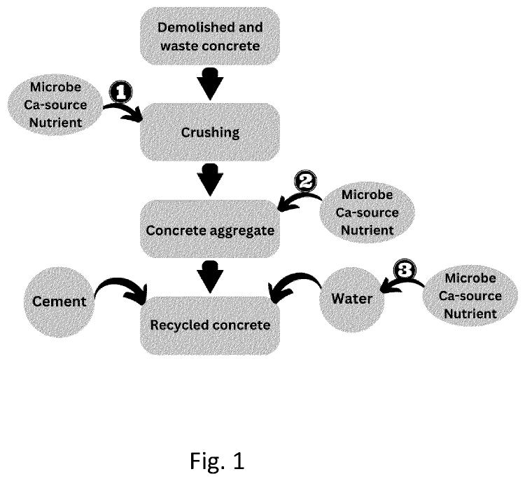

In FIG. 1 an exemplary concrete recycling process is illustrated. Shown are steps of the concrete recycling process starting from demolished and/or waste concrete (reclaimed concrete). Also indicated by numerals steps where microbial preparations can be added during the recycling. Position number 1 indicates the addition of microbial preparation right before or during concrete crushing. Position number 2 indicates the addition of microbial preparation to the aggregate right after crushing and sorting or during storage. The third position (3) depicts the addition of microbial preparation during concrete mixing and casting. These and other steps of the process are described further in the below.

The present invention also provides novel psychrophilic and alkaliphilic microbes that have mineralizing activity, i.e. the microbes are able to induce formation of calcium carbonate in their environment. These microbes, which are particularly useful in the methods described herein, have been isolated from samples of water and soil in and around natural and manmade alkaline environments in Sweden. The microbial strains grow well at low temperatures, i.e. below 20° C. The strains produce endospores and are able to grow in alkaline media (e.g. at pH 10) and deposit calcite.

The so-called microbially induced calcium carbonate precipitation (MICP) technology is founded on the ability of certain microbes to form mineral deposits through the production of carbonate (CO₃²⁻) by the microbes, which subsequently reacts with calcium ions (Ca²⁺) to generate CaCO₃ deposits. These deposits fill cracks and pores, thereby resulting in reinforcement of concrete. This reinforcement is in part due to the good adhesion and chemical stability of the CaCO₃ crystals, which has led to the wide use of MICP technology in the reinforcement and repair of concrete.

Common mineralizing microbes are bacteria, including oxidizing bacteria, sulphate solubilizing bacteria, denitrifying bacteria and ureolytic bacteria.

The mineralization and deposition of CaCO₃ that is induced by microbes depends on a number of factors, including the type of microbes (typically bacterial), the concentration of the microbes, the calcium source, pH, available nutrients, nucleation center and temperature.

Bacteria produce the necessary enzymes (such as urease and carbonic anhydrase) to convert the appropriate chemical compounds into the carbonate ions. These chemical activities change the microenvironment around the bacteria to favor formation of CaCO₃ deposits. Mineral deposition through urea hydrolysis is induced by certain bacterial strains through reactions catalyzed by the enzyme urease, catalyzing the hydrolysis of urea, and carbonic anhydrase, catalyzing the hydration of dissolved CO₂ to form HCO₃⁻, which ionizes to CO₃²⁻ under alkaline conditions. In the absence of urea as the CO₂ source, bacteria or other microbes catalyze the formation of CO₃²⁻ from dissolved CO₂.

In the absence of urea as a CO₂ source, the underlying chemical reactions of the mineralization process can be summarized by the following:
1) Gaseous CO₂ dissolves in water to form hydrated aqueous CO₂:

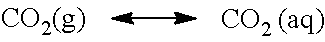

2) Hydrated aqueous CO₂ reacts with water to form H₂CO₃:

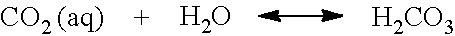

3) H₂CO₃ ionization in water generates H⁺ and HCO₃:

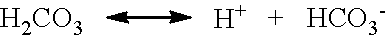

4) Under alkaline conditions, HCO₃ further ionizes to form CO₃²⁻ and H₂O:

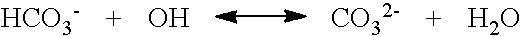 [SMILES: "HCO3-" ; O ; "CO32-" ; O]

5. In the presence of Ca²⁺, CaCO₃ precipitates are formed by reacting with CO₃²⁻:

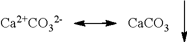

The surface charge of the microbes (typically bacteria) attracts calcium ions, the cells thereby serving as precipitation sites for formation of CaCO₃ crystals.

A major drawback of the use of microbes in production of recycled concrete aggregates is the limited temperature range of the microbes. In particular, microbes used to date in MICP have a mesophilic optimum, which means that their activity is highest at temperatures above 25° C. At low temperatures, including typical temperatures in colder climates such as in northern parts of the Northern Hemisphere, the microbes have no or minimal activity, rendering their use for the formation of mineral deposits minimal or nonexistent.

Experimentally it has been found that a temperature of about 30° C. is the optimum temperature for MICP using conventional (i.e. mesophilic) microbes.

The microbes disclosed herein are by contrast psychrophilic, i.e. the microbes are able to grow at low temperatures. As a result, the microbes can deposit CaCO₃ at low temperatures that are common in colder climates, thereby providing unique opportunities for use in the context of producing and storing recycled concrete.

The term “psychrophilic” as disclosed herein refers to microbes that have an optimal temperature for growth of 20° C. or lower.

Psychrophilic microbes can withstand temperatures as low as 0° C., meaning that the microbes are capable of growth at a temperature of close to 0° C. The microbes can withstand a temperature as high as 25° C.

Psychrophilic microbes as disclosed herein can accordingly grow at a temperature in the range of about 0° C. to about 25° C., including about 0° C. to about 20° C., about 4° C. to about 20° C. and about 10° C. to about 20° C. The optimum growth temperature of the microbes can be in the range of about 10° C. to about 25° C., such as in the range of about 10° C. to about 20° C. or about 15° C. to about 20° C.

The psychrophilic microbes are preferably also alkaliphilic, i.e. the microbes are capable of growth under alkaline conditions. Preferably, the microbes can grow at a pH in the range from about 8 to 13, such as about 8.5 to about 13, about 8.5 to about 12 or about 8.5 to about 11.

The microbes can be bacterial. For example the microbes can comprise one or more aerobic bacterial species, or the microbes can comprise a mixture of aerobic and anaerobic bacterial species. The bacteria can be from the species Bacillus.

For example, the bacteria can be from a species selected from Bacillus sp. 9A, Bacillus sp. 9D, Bacillus sp. 9E, Bacillus sp. B1, Bacillus sp. B5, Bacillus sp. CB1, Bacillus sp. CB2, Bacillus sp. 2B, Salipaludibacillus agaradhaerens D2.

The microbes can also be fungi, such as from a Fusarium sp, for example from fungal strain Fusarium sp CF7.

The microbes can preferably be selected from Planococcus sp with 16s rRNA sequence as set forth in SEQ ID NO:1, Alkalihalobacillus sp with 16s rRNA sequence as set forth in SEQ ID NO:2, Salinicoccus sp with 16s rRNA sequence as set forth in SEQ ID NO:3, Planococcus sp with 16s rRNA sequence as set forth in SEQ ID NO:4, Alkalihalophilus pseufofirmus with 16s rRNA sequence as set forth in SEQ ID NO:5, Shouchella patagoniensis with 16s rRNA sequence as set forth in SEQ ID NO:6, Alkalihalophilus pseufofirmus with 16s rRNA sequence as set forth in SEQ ID NO:7, or a species with 16s rRNA sequence that is at least 95%, at least 96%, at least 97%, at least 98%, at least 99%, at least 99.5%, at least 99.8% or at least 99.9% identical thereto.

In an embodiment, the microbes are from Shouchella patagoniensis with 16s rRNA sequence as set forth in SEQ ID NO:6, or from Shouchella patagoniensis with a 16s rRNA sequence that is at least 95%, at least 96%, at least 97%, at least 98%, at least 99%, at least 99.5%, at least 99.8% or at least 99.9% identical thereto.

The microbes can preferably have a 16s rRNA gene sequence (i.e. 16s rDNA sequence) that is at least 95%, at least 96%, at least 97%, at least 98%, at least 99%, at least 99.5%, at least 99.7%, at least 99.8% or at least 99.9% identical to a sequence selected from any one of SEQ ID NO: 1-7.

During growth under psychrophilic conditions, the microbes are capable of using nutrients and a source of calcium to form calcium carbonate under alkaline conditions.

The source of calcium can influence the type of calcium carbonate crystals that are formed. Typically, three forms of crystals can be formed, calcite, vaterite and aragonite. The first type, calcite, is considered the favored type for use in recycled concrete aggregates due to its high stability. The type of calcium source can influence the mineralization process.

The calcium source can be any inorganic salt of Ca²⁺. In certain embodiments, the salt is selected from calcium formate, calcium acetate, calcium lactate, calcium gluconate, calcium chloride and calcium nitrate. Other calcium sources include basic silicate rocks, such as basalt and dolerite, that are known to release calcium upon weathering. Thus, the calcium source can include one or more basic silicate rock, including basalt and dolerite. Such rocks can be ground or crushed to any desirable particle size prior to their use, i.e. mixing with recycled aggregate.

The calcium source can comprise two or more calcium sources, for example two or more inorganic salts of calcium, two or more basic silicate rocks, or any combination of two or more inorganic calcium salts and basic silicate rocks.

It is possible to combine psychrophilic and mesophilic microbes in the methods and compositions disclosed herein. Thus, the psychrophilic microbes disclosed herein can be combined with one or more mesophilic microorganism, i.e. microbes having an optimal temperature for growth at or above 25° C., such as about 30° C.

Any suitable nutrients can be used in the methods of composition disclosed herein. For growth, microbes need suitable nutrients, including carbon, nitrogen, oxygen and various trace elements. Suitable growth media are known in the art and can be used. Exemplary nutrients include yeast extract, peptone, aspartate, glutamate and trace elements. The nutrient can comprise trace elements and one or more selected from the group consisting of yeast extract, peptone, aspartate, and glutamate. The trace element can comprise one or more elements selected from the group comprising Zn, Co, Cu, Fe, Mn, Ni, B, P and Mo. For urease producing bacteria, the nutrients can further include urea.

The microbial preparation can be added in liquid or powder form during concrete mixing and/or casting.

Liquid compositions containing the mineralizing microbes can contain the microbes in a concentration in the range of 10⁴-10⁹ cfu/mL, such as in the range of 10⁵-10⁹ cfu/mL or in the range of 10⁶-10⁹ cfu/mL or in the range of 10⁷-10⁹ cfu/mL.

Concrete can be recycled by a process that involves the crushing and grading of reclaimed concrete to generate recycled concrete aggregate, sometimes also referred to as recycled aggregate. This recycled aggregate can be reused as-is or adapted for subsequent reuse followed by its use to prepare recycled concrete.

Recycled concrete aggregates can be produced by a two-stage process, where in a first step the concrete is crushed and in a second step the obtained aggregate is screened to remove contaminants and/or undesirable components such as reinforcement, paper, wood, plastics and gypsum. The screening can also include a step of sieving to select out particles within a desired size range.

The selecting can include separating recycled concrete particles within a desired size range from other particle sizes. The selection can be done using methods known in the art. Selected size ranges can for example be 4-8 mm, 8-16 mm, 10-20 mm 16-32 mm and 20-40 mm, although any size range can be selected. Thus, the lower limit of the size range can be from 2 to 20 mm. The upper limit of the size range can be from 16 to 50 mm.

For example, the recycled concrete can contain particles with a particle size that is less than 10 mm or a particles size that is between 10 and 20 mm.

The recycled concrete can also contain a mixture of particles within different size ranges. For example, a portion of the particles can have a particle size with a first small particle size, and a portion of the particles can have a particle size with a second larger particle size.

Demolished or crushed concrete contains a certain amount of mortar and cement paste from the original concrete that remains attached to the stone particles in the recycled aggregate. This attached mortar is believed to represent a main reason for the lower mechanical and chemical properties of recycled concrete aggregate compared with natural (fresh) aggregate used in fresh concrete.

Some of the differences in the properties of recycled concrete aggregate include increased water absorption, decreased bulk density, decreased specific gravity, increased abrasion loss, increased crushability, increased amount of dust particles, increased amount of organic impurities and potentially chemically harmful substances resulting from building demolition.

Thus, a challenge in the use of recycled aggregates to produce recycled concrete has been inferior mechanical and chemical properties of the recycled concrete. Incorporation of mineralizing microbes represents one manner in which the mechanical and chemical properties of the recycled concrete can be improved.

Concrete aggregate obtained through grinding or crushing of concrete can be treated with psychrophilic microbes as described herein. The treatment can occur during or following the crushing. Thus, the psychrophilic microbes can be added to the concrete during its crushing or grinding, or the psychrophilic microbes can be added to the aggregate after the crushing or grinding.

The microbes can be added in dry or liquid form, i.e. as a liquid or as a dry composition. The microbes can be added in combination with one or more source of calcium, one or more source of CO₂ and one or more nutrients.

The one or more source of CO₂ can at least in part be atmospheric CO₂. Typically, the main source of CO₂ is a nutrient such as lactic acid, which may also be used as a source of calcium (as calcium lactate). For ureolytic microbes, i.e. microbes producing urease, the one or more source of CO₂ can be urea.

When provided in dry form, the microbes can be added as a dry composition that includes one or more source of calcium, one or more source of CO₂ and/or one or more nutrient to the recycled concrete aggregate during its crushing or grinding. Alternatively, the microbes can be added as a dry composition after the crushing or grinding. The recycled aggregate can be sieved or graded to select certain aggregate sizes before the addition of the microbes.

When provided in liquid form, the microbes can be added as a liquid composition that contains the microbes and one or more source of calcium, one or more source of CO₂ and/or one or more nutrient. The liquid can be added to the recycled aggregate by spraying onto the aggregate. Alternatively, the liquid can be added by immersing the recycled aggregate in a liquid composition containing the microbes and the one or more source of calcium, one or more source of CO₂ and/or one or more nutrient.

The recycled concrete aggregates disclosed herein have improved chemical and mechanical properties over recycled concrete aggregates that do not contain psychrophilic mineralizing microbes. For example, the recycled concrete aggregates have reduced water absorption properties compared with untreated aggregates, i.e. aggregates that do not contain psychrophilic mineralizing microbes.

Further, the recycled concrete aggregates have improved properties during storage. For example, the recycled concrete aggregate disclosed herein can result in reduced water absorption compared with ordinary recycled aggregate. The reduction in water absorption can be in the range of 5 to 20%, such as in the range of 10 to 20%. The reduction in water absorption can be at least 5%, at least 10% or at least 15%. The recycled concrete aggregate may also have additional favorable properties such as decreased clumping and/or increased flowability.

Like any other building materials, sorted out recycled concrete aggregate is often stored before its use. Calcite precipitating microbial preparations can be sprayed over or on the aggregate, mixed and stored. During this storage time, the microbes deposit calcite on the recycled aggregate surface and improve its performance property when used in making concrete.

Alternatively, the aggregate can be immersed in liquid preparation containing calcite precipitating microbe or microbes, one or more calcium source and nutrients for up to 3-4 weeks. The microbes deposit calcite on the surface of the recycled aggregate during the immersion. The microbes are expected to have better aeration which allows them to deposit calcite effectively. Moreover, the recycled aggregate thus treated can be ready to use like virgin aggregate in mixing and casting concrete. The adsorbed microbes will continue to deposit calcite if fresh calcium, carbon and other nutrients are added during mixing. This provides a vast improvement over current recycled concrete in terms of its usefulness for preparing new concrete, which typically must contain a large proportion of virgin (fresh) concrete aggregate and only supplemented by recycled aggregate.

Accordingly, further provided is a method of improving the properties of recycled concrete aggregates during storage at temperatures below 20° C., the method comprising adding a composition comprising at least one alkaliphilic and psychrophilic microbial culture capable of depositing calcium carbonate on the recycled concrete aggregate.

When used, the recycled concrete aggregate described herein can be mixed into fresh concrete. The term “fresh concrete” in this context refers to concrete that contains “fresh” aggregate, i.e. aggregate that does not originate from concrete and is thus not recycled. The term can also be referred to as “natural concrete”, which may accordingly comprise “natural” aggregate. Accordingly, also provided is recycled concrete containing a mixture of fresh concrete and recycled concrete aggregates that contains at least one psychrophilic microbial species capable of inducing formation of CaCO₃. The recycled aggregate can also be mixed with fresh (virgin) aggregate and subsequently used for preparing concrete.

Alternatively, recycled concrete aggregate that has been mixed with fresh aggregate can be strengthened using the microbes described herein. Thus, another application relates to a method of strengthening such mixtures of fresh concrete and recycled concrete aggregate, the method comprising immersing concrete comprising a mixture of recycled concrete aggregate and fresh concrete in a liquid composition comprising at least one alkaliphilic and psychrophilic microbe capable of depositing calcium carbonate in the concrete. The ratio of recycled concrete aggregate to fresh aggregate can generally be in the range of 1:10 to 10:1 (w:w). For example, the ratio can be in the range of 1:10 to 5:1, in the range of 1:5 to 5:1, in the range of 1:4 to 4:1, in the range of 1:3 to 3:1, in the range of 1:2 to 2:1 or in the range of 1:1.5 to 1.5:1, or the ratio can be about 1:1. The ratio of recycled concrete aggregate to fresh aggregate can preferably be less than 1:1, the resulting recycled concrete contains less than 50% by weight, such as less than 40%, less than 30%, less than 20% or less than 10% by weight, of fresh aggregate.

It is also possible to use the recycled concrete aggregate disclosed herein exclusively to prepare concrete, i.e. without supplying any fresh or virgin aggregate. Thereby fresh concrete prepared by using recycled aggregate only is possible.

The methods described herein can be extended to multiple treatment steps, where in each treatment step recycled concrete aggregate or concrete containing recycled concrete aggregate is treated two or more times with a composition containing psychrophilic mineralizing (i.e., capable of inducing calcium carbonate formation) microbes. Such microbes can be identical or different in the treatment steps. In other words, one treatment step can include treatment with one microbial strain, and subsequent step(s) include treatment with one or more different microbial strains. The strains can have different properties, for example one strain can be a psychrophilic strain and another strain can be mesophilic. This way, the treated recycled concrete aggregate or concrete contains microbes capable of inducing mineralization across a range of temperatures, such as from about 5° C. to 30° C. or higher.

Each treatment step in such series of treatments steps can also include treatment with one or more microbial strains that can be identical or different in the treatment steps.

The treatment can further include one or more steps of adding microbes in a dry form in one step, followed by one or more subsequent steps of adding one or more additional microbe in liquid form.

It should be noted that the microbial species provided in dry form can comprise lyophilized vegetative cells or dried microbial (e.g., bacterial) spores. Hence, the microbial material is selected from the group consisting of a microbe, a lyophilized microbe and a microbial spore of a microbe. In a liquid form, the microbial material can be selected from the group consisting of a microbe and a microbial spore of a microbe.

As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and claims, the terms “comprise”, “including”, “having”, and “contain” and their variations should be understood as meaning “including but not limited to” and are not intended to exclude other components.

Those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure are used. The present invention also covers the exact terms, features, values and ranges etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

The term “at least one” should be understood as meaning “one or more”, and therefore includes both embodiments that include one or multiple components. Furthermore, dependent claims that refer to independent claims that describe features with “at least one” have the same meaning, both when the feature is referred to as “the” and “the at least one”.

Features disclosed in the specification, unless stated otherwise, can be replaced by alternative features serving the same, equivalent or similar purpose. Thus, unless stated otherwise, each feature disclosed represents one example of a generic series of equivalent or similar features.

Use of exemplary language, such as “for instance”, “such as”, “for example” and the like, is merely intended to better illustrate the invention and does not indicate a limitation on the scope of the invention unless so claimed. Any steps described in the specification may be performed in any order or simultaneously, unless the context clearly indicates otherwise.

All of the features and/or steps disclosed in the specification can be combined in any combination, except for combinations where at least some of the features and/or steps are mutually exclusive. In particular, preferred features of the invention are applicable to all aspects of the invention and may be used in any combination. The scope of the disclosure is only limited by the appended patent claims.

The invention is further described by the following non-limiting examples.

### Example 1

Microbes were isolated around Skåne, in southern Sweden. The pH of the samples was in the range of 8 to 9.5 at the time of sampling. About 1 ml of liquid samples and 1 μm of soil samples were added to flasks containing sterile medium composed of 0.5% yeast extract 0.5% calcium lactate, 0.01 MgSO4. The pH of the enrichment medium was 10. After 3 days shaking (150 rpm) at 12° C., cultures were transferred to agar plates composed of the same ingredients used in the liquid (submerged) cultivation but containing 1.5% agar and incubated at 10° C. After one week, colonies were picked and transferred to new plates. Pure colonies were tested for calcite production both in solid and liquid medium. Ten isolates that grow relatively faster, and deposit copious amount of calcite were selected.

The bacterial strains are rod or coccus shaped, aerobic, many of them are endospore forming, able to grow lavishly within 3 days both in liquid and solid media at pH 10 and temperature of 10° C. The selected bacterial isolates 9A, 9D, 9E, B1, B5, CB1, CB2, 2B, 2D and unidentified fungal strain CF7. Further detailed characterization of the strains has revealed their proper identity, see Example 4, in particular for strains B1, CB1, CB2, 2B.

### Example 2

Psychrophilic alkaliphilic bacteria grown at 12° C. for 3 days in orbital incubator shaker were harvested by centrifugation and added to concrete mix to a final load of about 10⁶ CFU/mL. The microbes, calcium lactate (final concentration in concrete, 5 μm/L and yeast extract (final concentration 3 μm/L) were resuspended/dissolved in water used to make the concrete. The concrete mix proportion per cubic meter was 380 kg cement, 602 kg fine aggregate, 1085 kg recycled coarse aggregate and 190 liter water containing the microbe, calcium lactate and yeast extract. The concrete after 5 min mixing with a mixer, poured to plastic mold. The concrete was taken out of the mold after 48 hr and kept for 3 weeks in a room with average temperature of about 10° C.

### Example 3

For a water absorption test, concrete was prepared as described in Example 2. The control concrete was prepared exactly in the same way as in Example 2 but without adding microbes. The test specimen contained the microbes. After 3 weeks of casting, the concrete structures were dried in an oven (60° C.) until their weight is constant. Then the concrete was soaked for 6 hours in water, dried (wiped) with tissue paper and left overnight on the bench. Then, the concrete weight was measured. For each concrete structure, the amount of water absorbed is obtained by deducting its dry weight from the soaked weight. The percentage in reduction of water absorption was calculated as:

$$\% absorption reduction \frac{Amountabsorbed bycontrol - Amountabsorbed bytest ) \times 100}{Amountabsorbed bycontrol}$$

The reduction in water observation by the microbes varies. The best absorption reduction was observed when isolate Bacillus 9D is used and the least was achieved when Bacillus CB2 was used (Table 1).

TABLE 1
Water absorption reduction of RCE prepared with various isolates

| Isolate | Water absorption reduction |
|---|---|
| 9A | 15% |
| 9D | 17% |
| 9E | 16% |
| B1 | 13% |
| B5 | 6% |
| CB1 | 7% |
| CB2 | 5% |
| 2B | 6% |
| 2D | 10% |
| CF7 (fungal isolate) | 8% |

### Example 4. Strain Characteristics

Isolated strains were characterized, including by 16s rRNA sequencing, to further characterize the strains, with the SEQ ID referring to the respective rRNA gene sequence.

The following Table 2 shows the biological and physiochemical characteristics of isolated strains:

TABLE 2A
Strain principal characteristics

| Isolate | Description | SEQ ID |
|---|---|---|
| Bf1 | Gram-positive, psychrotolerant, halophilic | 1 |
| | bacterium, non-sporulating | |
| CB1 | Obligate aerobe, spore-forming, psychrophilic | 2 |
| | alkaliphilic bacterium | |
| CB2 | Halophilic, alkaliphilic, Psychrotolerant | 3 |
| | non-motile, obligately aerobic Gram-positive | |
| B1 | Gram-positive, psychrotolerant, halophilic, | 4 |
| | non-sporulating, motile bacterium | |
| 6A | Aerobe, Gram-positivem spore forming bacterium | 5 |
| 2B | Gram-positive, rod-shaped, spore-forming bacterium | 6 |
| S2 | Aerobe, Gram-positive spore forming bacterium | 7 |

TABLE 2B
Strain biological characteristics

| Isolate | Genus/Species | Temp range | pH range | SEQ ID |
|---|---|---|---|---|
| Bf1 | Planococcus sp. | 7-20° C. | 8-10.5 | 1 |
| CB1 | Alkalihalobacillus | 5-25° C. | 8-11 | 2 |
| | sp. | | | |
| CB2 | Salinicoccus sp. | 7-28° C. | 7-10.5 | 3 |
| B1 | Planococcus sp. | 5-25° C. | 8-10.5 | 4 |
| 6A | Alkalihalophilus | 5-30° C. | 8-11 | 5 |
| | pseudofirmus 6A | | | |
| 2B | Shouchella | 5-20° C. | 8-11 | 6 |
| | patagonienis 2B | | | |
| S2 | Alkalihalophilus | 5-30° C. | 8-11 | 7 |
| | pseudofirmus S2 | | | |

As can be seen, upon 16s rRNA gene sequencing, the isolates B1, CB2 and 2B were found to be species from respectively Planococcus, Salinicoccus and Shouchella and not Bacillus.

Furthermore, isolate D2 was identified by 16s rRNA sequencing to be from Salipaludibacillus agaradhaerens, and fungal isolate CF7 was identified by 16s rRNA sequencing to be a Fusarium sp.

### Example 5. Sequence Comparison

Sequence comparison was performed to determine the similarities between the sequenced species and known species. As can be seen in the following, the isolated and sequenced strains are most likely new species, with Shouchella patagoniensis 2B, Alkalihalophilus pseudofirmus 6A and Alkalihalophilus pseudofirmus S2 being most similar to known species.

Planococcus sp. Bf1

| Species | Percent identity |
|---|---|
| Planococcus citreus | 99.57% |
| Planococcus rifietoensis | 99.57% |
| Planococcus dechangensis | 99.36% |
| Planococcus maritimus | 99.14% |

Planococcus sp. B1

| Species | Percent identity |
|---|---|
| Planococcus citreus | 99.79% |
| Planococcus rifietoensis | 99.72% |
| Planococcus dechangensis | 98.89% |
| Planococcus maritimus | 98.72% |

### Alkalihalobacillus sp.CB1

| Species | Percent identity |
|---|---|
| Alkalihalobacillus | 98.32% |
| alcalophilus | |
| Alkalihalobacillus | 98.18% |
| alcalophilus | |
| Alkalihalobacillus | 97.48% |
| trypoxylicola | |
| Alkalihalobacillus | 97.13% |
| pseudalcaliphilus | |

Salinicoccus sp. CB2

| Species | Percent identity |
|---|---|
| Salinicoccus salitudinis | 97.97% |
| Lacicoccus kunmingensis | 96.79% |
| Salinicoccus qingdaonensis | 96.95% |
| Salinicoccus kekensis | 95.26% |

Alkalihalophilus pseudofirmus 6A

| Species | Percent identity |
|---|---|
| Alkalihalophilus | 99.93% |
| pseudofirmus | |
| Alkalihalophilus | 98.67% |
| lindianensis | |
| Halalkalibacter | 97.05% |
| nanhaiisediminis | |
| Halalkalibacter akibai | 96.46% |

Alkalihalophilus pseudofirmus S2

| Species | Percent identity |
|---|---|
| Alkalihalophilus pseudofirmus | 99.79% |
| Alkalihalophilus lindianensis | 98.52% |
| Halalkalibacter | 96.96% |
| nanhaiisediminis | |
| Halalkalibacter akibai | 96.28% |

### Example 6. Physiochemical Properties of Recycled Concrete Aggregates

### Preparation of Samples

Recycled aggregate was separated into fine aggregate (<10 mm) and coarse aggregate (10-20 mm). The recycled fine aggregate was used to substitute natural fine and similarly the recycled aggregate was used to replace natural aggregate. Mortar samples were prepared by mixing 310 g fine aggregate (natural or recycled), 340 g coarse aggregate (natural or recycled), 150 g cement and 75 g water with or without CaCO₃ precipitating microbial preparation. The water/cement ratio was kept at 0.5. The bacterial preparation had 10⁶ CFU per ml.

The effect of the microbial based preparation on water absorption, porosity, density and compressional strength of the concrete structures were analyzed.

Three mortar preparations were made: “Recycled Fine” contained recycled fine aggregate and coarse natural aggregate; “Recycled coarse” contained recycled coarse aggregate and natural fine aggregate; “Recycled fine and coarse” contained both fine and course recycled aggregate (only, no natural aggregate).

### Water Absorption

Water absorption was measured as described in Example 3, with the difference that the soak time was 24 hours.

$$The water absorption was calculated based on A = ( \frac{W 1 - W 0}{W 0} )$$

where A is water absorption, W1 is weight of water saturated concrete (soaked in water for 24 h), W0 is the dry weight of the concrete.

TABLE 3
Water absorption reduction after 21 days of curing at 8° C. The
water absorption of the controls for recycled fine, aggregate, and
fine and recycled were 9.3, 7.7 and 15%, respectively and these values
are taken as 100% for each of the three combination studies.

| | Water absorption reduction (%) | | |
|---|---|---|---|
| | | | Recycled |
| | Recycled | Recycled | fine and |
| Species | Fine | coarse | coarse |
| Planococcus sp. Bf1 | 9.6 | 9.1 | 10.4 |
| Alkalihalobacillus sp. CB1 | 18.1 | 14.3 | 15.7 |
| Salinicoccus sp. CB2 | 27.4 | 12.1 | 19.3 |
| Planococcus sp. B1 | 8.4 | 6.8 | 9.7 |
| Alkalihalophilus pseudofirmus 6A | 13.3 | 8.4 | 10.0 |
| Shouchella patagoniensis 2B | 7.3 | 5.4 | 6.2 |
| Alkalihalophilus pseudofirmus S2 | 12.5 | 9.7 | 11.0 |

### Porosity

Porosity is the measure of voids in a concrete. It has a significant impact on the durability and performance of concrete structures. In most cases, it is desired the concrete to have low porosity. The porosity is calculated using this formula:

$$Porosity = ( \frac{Void volume}{Total solid volume} ) \times 100$$

The water accessible void volume of the concrete is obtained by deducting its dry weight from its water saturated weight. The dry weight is measured by drying the concrete in oven until its weight is constant. The wet weight is determined by soaking the concrete in water for 24 h followed by removing from the water and wiping the excess water from the surface and weigh it. The total volume of the concrete is measured by immersing the wet concrete in water and taking the increase in volume as the volume of the concrete

TABLE 4
The porosity of concrete samples after 21 days of curing at 8° C.

| | Porosity (%) | | |
|---|---|---|---|
| | | Recycled | Recycled |
| | Recycled | coarse | fine and |
| Species | Fine | aggregate | aggregate |
| Control | 14.21 | 11.20 | 16.32 |
| Planococcus sp. Bf1 | 13.35 | 10.52 | 14.83 |
| Alkalihalobacillus sp. CB1 | 12.75 | 10.45 | 13.53 |
| Salinicoccus sp. CB2 | 11.30 | 9.70 | 12.41 |
| Planococcus sp. B1 | 13.21 | 10.81 | 14.78 |
| Alkalihalophilus pseudofirmus 6A | 12.34 | 10.48 | 13.28 |
| Shouchella patagoniensis 2B | 13.94 | 11.07 | 14.92 |
| Alkalihalophilus pseudofirmus S2 | 12.55 | 10.39 | 13.36 |

### Density

Density is the measure of solid mass of concrete per unit volume. Often, the denser the concrete, the higher the performance. The density of the concrete is calculated based on the formula:

$$ρ = \frac{m}{V}$$

where ρ stands for density, m is the dry mass of the concrete and Vis the total volume of the dry concrete. The total volume is obtained by deducting the void volume from the total volume of the concrete.

TABLE 5
The density of the concrete samples after 21 days of curing at 8° C.

| | Density (gm/cm³) | | |
|---|---|---|---|
| | | Recycled | Recycled |
| | Recycled | coarse | fine and |
| Species | Fine | aggregate | aggregate |
| Control | 2.48 | 2.66 | 2.14 |
| Planococcus sp. Bf1 | 2.47 | 2.63 | 2.14 |
| Alkalihalobacillus sp. CB1 | 2.56 | 2.70 | 2.21 |
| Salinicoccus sp. CB2 | 2.68 | 2.71 | 2.27 |
| Planococcus sp. B1 | 2.45 | 2.58 | 2.15 |
| Alkalihalophilus pseudofirmus 6A | 2.50 | 2.68 | 2.20 |
| Shouchella patagoniensis 2B | 2.43 | 2.65 | 2.17 |
| Alkalihalophilus pseudofirmus S2 | 2.51 | 2.67 | 2.19 |

### Compressive Strength

The compressive strength is estimated using rebound hammer test. The average rebound value (R) obtained from the hammer testing is matched to the corresponding compressive strength value on the conversion chart which is given in MPa.

TABLE 6
Compressive strength after 21 days of curing at 8° C. The
compressivel strength of the concrete made of natural aggregates
(both fine and aggregate) was about 38 MPa after 21 days.

| | Compressive strength (MPa) | | |
|---|---|---|---|
| | | Recycled | Recycled |
| | Recycled | coarse | fine and |
| Species | Fine | aggregate | aggregate |
| Control | 28 | 32 | 27 |
| Planococcus sp. Bf1 | 29 | 33 | 28 |
| Alkalihalobacillus sp. CB1 | 31 | 36 | 29 |
| Salinicoccus sp. CB2 | 34 | 40 | 35 |
| Planococcus sp. B1 | 29 | 31 | 28 |
| Alkalihalophilus pseudofirmus 6A | 30 | 34 | 31 |
| Shouchella patagoniensis 2 | 29 | 32 | 29 |
| Alkalihalophilus pseudofirmus S2 | 31 | 35 | 30 |

1. A method of recycling reclaimed concrete comprising crushing or grinding reclaimed concrete to obtain concrete aggregates, wherein at least one composition comprising an alkaliphilic and psychrophilic microbe capable of depositing calcium carbonate is added to the reclaimed concrete during or after the crushing or grinding, thereby obtaining a recycled concrete aggregate comprising at least one alkaliphilic and psychrophilic microbial culture.

2. The method of claim 1, wherein the at least one composition is added in a liquid or dry form.

3. The method of claim 1, wherein the at least one composition is added in a dry form during or after the crushing or grinding.

4. The method of claim 1, wherein the at least one composition is added in liquid form during or after the crushing or grinding.

5. The method of claim 4, wherein the at least one composition is added by spraying the composition onto the recycled concrete aggregate after the crushing or grinding.

6. The method of claim 4, wherein the at least one composition is added by immersing the concrete aggregate in the composition in liquid form.

7. The method of claim 1, wherein the at least one microbe further comprises at least one mesophilic microbe capable of depositing calcium carbonate.

8. The method of claim 1, further comprising processing the recycled concrete aggregate to one or more sieving steps to obtain aggregate containing particles within a predetermined size range.

9. The method of claim 1, wherein the at least one alkaliphilic and psychrophilic microbe is selected from Planococcus sp with 16s rRNA sequence as set forth in SEQ ID NO:1, Alkalihalobacillus sp with 16s rRNA sequence as set forth in SEQ ID NO:2, Salinicoccus sp with 16s rRNA sequence as set forth in SEQ ID NO:3, Planococcus sp with 16s rRNA sequence as set forth in SEQ ID NO:4, Alkalihalophilus pseufofirmus with 16s rRNA sequence as set forth in SEQ ID NO: 5, Shouchella patagoniensis with 16s rRNA sequence as set forth in SEQ ID NO: 6, Alkalihalophilus pseufofirmus with 16s rRNA sequence as set forth in SEQ ID NO:7, or a species with 16s rRNA sequence that is at least 95%, at least 96%, at least 97%, at least 98%, at least 99%, at least 99.5%, at least 99.8% or at least 99.9% identical thereto.

10. The method of claim 1, further comprising mixing or blending the recycled concrete aggregate with fresh aggregate.

11. Recycled concrete aggregate characterized in that the aggregate comprises at least one culture comprising alkaliphilic and psychrophilic microbes that are capable of inducing calcium carbonate deposition.

12. The recycled concrete aggregate of claim 11, wherein the aggregate comprises in the range of 0.1 to 15% by weight of the at least one culture.

13. The recycled concrete aggregate of claim 11, further comprising at least one calcium source, at least one CO₂ source and one or more nutrients.

14. The recycled concrete aggregate of claim 11, wherein the at least one alkaliphilic and psychrophilic microbial culture is selected Planococcus sp with 16s rRNA sequence as set forth in SEQ ID NO:1, Alkalihalobacillus sp with 16s rRNA sequence as set forth in SEQ ID NO:2, Salinicoccus sp with 16s rRNA sequence as set forth in SEQ ID NO:3, Planococcus sp with 16s rRNA sequence as set forth in SEQ ID NO: 4, Alkalihalophilus pseufofirmus with 16s rRNA sequence as set forth in SEQ ID NO:5, Shouchella patagoniensis with 16s rRNA sequence as set forth in SEQ ID NO:6, Alkalihalophilus pseufofirmus with 16s rRNA sequence as set forth in SEQ ID NO:7, or a species with 16s rRNA sequence that is at least 95%, at least 96%, at least 97%, at least 98%, at least 99%, at least 99.5%, at least 99.8% or at least 99.9% identical thereto.

15. The recycled concrete aggregate of claim 1, further comprising at least one mesophilic microbial culture that is capable of inducing calcium carbonate deposition.

16. (canceled)

17. (canceled)

18. (canceled)

19. (canceled)

20. (canceled)

21. (canceled)

22. (canceled)

23. (canceled)

24. (canceled)

25. A microbial strain capable of inducing deposition of CaCO₃, wherein the microbial strain is active under alkaline conditions and a temperature below 20° C., preferably 5-15° C., wherein the microbial strain is from a Planococcus sp with 16s rRNA sequence as set forth in SEQ ID NO:1, Alkalihalobacillus sp with 16s rRNA sequence as set forth in SEQ ID NO:2, Salinicoccus sp with 16s rRNA sequence as set forth in SEQ ID NO:3, Planococcus sp with 16s rRNA sequence as set forth in SEQ ID NO:4, Alkalihalophilus pseufofirmus with 16s rRNA sequence as set forth in SEQ ID NO: 5, Shouchella patagoniensis with 16s rRNA sequence as set forth in SEQ ID NO: 6, Alkalihalophilus pseufofirmus with 16s rRNA sequence as set forth in SEQ ID NO:7, or a species with 16s rRNA sequence that is at least 95%, at least 96%, at least 97%, at least 98%, at least 99%, at least 99.5%, at least 99.8% or at least 99.9% identical thereto.

26. The microbial strain of claim 25, wherein the microbial strain is from a Planococcus sp with 16s rRNA sequence as set forth in SEQ ID NO:1, Alkalihalobacillus sp with 16s rRNA sequence as set forth in SEQ ID NO:2, Salinicoccus sp with 16s rRNA sequence as set forth in SEQ ID NO:3, Planococcus sp with 16s rRNA sequence as set forth in SEQ ID NO:4, Alkalihalophilus pseufofirmus with 16s rRNA sequence as set forth in SEQ ID NO: 5, Shouchella patagoniensis with 16s rRNA sequence as set forth in SEQ ID NO: 6, Alkalihalophilus pseufofirmus with 16s rRNA sequence as set forth in SEQ ID NO:7.


SEQUENCE LISTING (7 sequences)

SEQ ID NO: 1 - DNA, 1396 bp, Planococcus sp.
  source 1..1396: mol_type = genomic DNA
ggagcttgctccattggtttagcggcggacgggtgagtaacacgtgggcaacctgccctgcagatcgggataactccgggaaaccggtgctaataccgaatagtttgcggcctctcatgaggccacacggaaagacggtttcggctgtcactgcaggatgggcccgcggcgcattagctagttggtgaggtaacggctcaccaaggccacgatgcgtagccgacctgagagggtgatcggccacactgggactgagacacggcccagactcctacgggaggcagcagtagggaatcttccgcaatggacgaaagtctgacggagcaacgccgcgtgagtgaagaaggttttcggatcgtaaaactctgttgtgagggaagaacaagtaccaattaactactggtaccttgacggtacctcaccagaaagccacggctaactacgtgccagcagccgcggtaatacgtaggtggcaagcgttgtccggaattattgggcgtaaagcgcgcgcaggcggtcctttaagtctgatgtgaaagcccacggctcaaccgtggagggtcattggaaactgggggacttgagtgcagaagaggaaagtggaattccacgtgtagcggtgaaatgcgtagagatgtggaggaacaccagtggcgaaggcgactttctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcccgctgaccgcctaggagactaggctttcccttcggggacagcggtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacggtacaaagggctgcaaacccgcgagggggagccaatcccagaaaaccgttctcagttcggattgcaggctgcaactcgcctgcatgaagccggaatcgctagtaatcgcggatcagcatgccgcggtgaatacgttcccgggccttgtacacaccgcccgtcacaccacgagagtttgtaacacccgaagtcggtggggtaacccttacgggagccagcc

SEQ ID NO: 2 - DNA, 1427 bp, Alkalihalobacillus sp.
  source 1..1427: mol_type = genomic DNA
catgcaagtcgagcggactaaagggagcttgctcccagaggttagcggcggacgggtgagtaacacgtgggcaacctgccctgtagactgggataacatcgagaaatcggtgctaataccggataatcattggagcttcatggttctaatgtaaaagatggctccggctatcactatgggatgggcccgcggcgcattagctagttggtaaggtaatggcttaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccattcaaatagggtggcaccttgacggtacctaaccagaaagccacggctaactacgtgccagcagccgcggtaatacgtaggtggcaagcgttgtccggaattattgggcgtaaagcgcgcgcaggcggtcttttaagtctgatgtgaaatctcggggctcaaccccgagcggtcattggaaactgggagacttgagtacagaagaggagagtggaattccacgtgtagcggtgaaatgcgtagatatgtggaggaacaccagtggcgaaggcgactctctggtctgtaactgacgctgaggcgcgaaagtgtggggagcaaacaggattagataccctggtagtccacaccgtaaacgatgagtgctaggtgttaggggtttcgatgcccttagtgccgaagttaacacattaagcactccgcctggggagtacggccgcaaggctgaaactcaaaggaattgacgggggcccgcacaagcagtggagcatgtggtttaattcgaagcaacgcgaagaaccttaccaggtcttgacatcctttgaccactctagagatagagctttccccttcgggggacaaagtgacaggtggtgcatggttgtcgtcagctcgtgtcgtgagatgttgggttaagtcccgcaacgagcgcaacccttgatcttagttgccagcattaagttgggcactctaaggtgactgccggtgacaaaccggaggaaggtggggatgacgtcaaatcatcatgccccttatgacctgggctacacacgtgctacaatggatggtacaaagggcagcaaaaccgcgaggtcgagccaatcccataaagccattctcagttcggattgtaggctgcaactcgcctacatgaagccggaattgctagtaatcgcggatcagcatgccgcggtgaatacgttcccgggccttgtacacaccgcccgtcacaccacgagagtttgtaacacccgaagtcggtggagtaacctttggaggctagccgcctaagg

SEQ ID NO: 3 - DNA, 1432 bp, Salinicoccus sp.
  source 1..1432: mol_type = genomic DNA
tcctatacatgcaagtcgagcgcaaagatcaggagcttgctcctgtgatttgagcggcggacgggtgagtaacacgtaggcaacctacccatcagactgggataaccacgggaaaccgtggctaataccggatgancctttgagacannagtctcaaagttgaaaggcgcttttggtgccgttgatggatgggcctgcggcgcattagctagttggtgagatagtggctcaccaaggcgacgatgcgtagccgacctgagagggtgatcggccacactgggactgagacacggcccagactcctacgggaggcagcagtagggaatcttccgcaatggacgcaagtctgacggagcaacgccgcgtgagtgaagaagggtttcggctcgtaaaactctgttgttaaggaagaatgacacatggagtaactgccatgtgtgtgacggtacttaaccagaaagccacggctaactacgtgccagcagccgcggtaatacgtaggtggcaagcgttatccggaattattgggcgtaaagcgcgcgtaggcggtttcttaagtctgatgtgaaagcccccggctcaaccggggagggtcattggaaactggggaacttgagtgcagaagaggagagtggaattccatgtgtagcggtgaaatgcgcagagatatggaggaacaccagtggcgaaggcggctctctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgaccgccatggagacatggcttcccttcggggcagagtgacaggtggtgcatggttgtcgtcagctcgtgtcgtgagatgttgggttaagtcccgcaacgagcgcaacccttaaacttagttgccagcattcagttgggcactctaagttgactgccggtgacaaaccggaggaaggtggggatgacgtcaaatcatcatgccccttatgatttgggctacacacgtgctacaatggacaggttacaaagggcagcgaagccgcaaggccaagcgaatcccataaaactgttctcagttcggattggagtctgcaactcgactccatgaagctggaatcgctagtaatcgtggatcagcatgccacggtgaatacgttcccgggtcttgtacacaccgcccgtcacaccacggaagtcagtaacacctgaagccggtgggccaaccgttatggaggcagccgtcgaag

SEQ ID NO: 4 - DNA, 1432 bp, Planococcus sp.
  source 1..1432: mol_type = genomic DNA
ctaatacatgcaagtcgagcggagatartggagcttgctccattatcttagcggcggacgggtgagtaacacgtgggcaacctgccctgcagatcgggataactccgggaaaccggtgctaataccgaatagtttgcggcctctcatgaggctgcacggaaagacggtttcggctgtcactgcaggatgggcccgcggcgcattagctagttggtggggtaacggctcaccaaggccacgatgcgtagccgacctgagagggtgatcggccacactgggactgagacacggcccagactcctacgggaggcagcagtagggaatcttccgcaatggacgaaagtctgacggagcaacgccgcgtgagtgaagaaggttttcggatcgtaaaactctgttgtgagggaagaacaagtaccaactaactactggtaccttgacggtacctcaccagaaagccacggctaactacgtgccagcagccgcggtaatacgtaggtggcaagcgttgtccggaattattgggcgtaaagcgcgcgcaggcggtcctttaagtctgatgtgaaagcccacggctcaaccgtggagggtcattggaaactgggggacttgagtgcagaagaggaaagtggaattccacgtgtagcggtgaaatgcgtagagatgtggaggaacaccagtggcgaaggcgactttctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcccgctgaccgcctaggagactaggctttcccttcggggacagcggtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacggtacaaagggctgcaaacccgcgagggggagccaatcccagaaaaccgttctcagttcggattgcaggctgcaactcgcctgcatgaagccggaatcgctagtaatcgcggatcagcatgccgcggtgaatacgttcccgggccttgtacacaccgcccgtcacaccacgagagtttgtaacacccgaagtcggtggggtaacccttacgggagccagccgccgaag

SEQ ID NO: 5 - DNA, 1431 bp, Alkalihalophilus pseudofirmus
  source 1..1431: mol_type = genomic DNA
catgcaagtcgagcggactgatgggagcttgctccctgatgttagcggcggacgggtgagtaacacgtgggcaacctgcctgtaagactgggataactccgggaaaccggggctaataccggataacccgttccacctcatggtggagcggtaaaagatggcctctggctatcacttacagatgggcccgcggcgcattagctagttggtaaggtaacggcttaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgtttgaataaggcggcaccttgacggtacctaaccagaaagccacggctaactacgtgccagcagccgcggtaatacgtaggtggcaagcgttgtccggaattattgggcgtaaagcgcgcgcaggcggtctcttaagtctgatgtgaaagcccacggctcaaccgtggagggtcattggaaactgggagacttgagtacagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccttagtgccgaagttaacacattaagcactccgcctggggagtacgaccgcaaggttgaaactcaaaggaattgacgggggcccgcacaagcagtggagcatgtggtttaattcgaagcaacgcgaagaaccttaccaggtcttgacatcctttgaccactctagagatagagctttccccttcgggggacaaagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaaaaccgcgaggttgagcgaatcccataaagccattctcagttcggattgtaggctgcaactcgcctacatgaagccggaattgctagtaatcgcggatcagcatgccgcggtgaatacgttcccgggccttgtacacaccgcccgtcacaccacgagagtttgtaacacccgaagtcggtgaggtaaccttttggagccagccgcctaaggtgg

SEQ ID NO: 6 - DNA, 1436 bp, Shouchella patagoniensis
  source 1..1436: mol_type = genomic DNA
atgcctaatacatgcaagtcgagcggacagaagggagcttgctcctggaagtcagcggcggacgggtgagtaacacgtgggcaacctgccccttagactgggataacttcgggaaaccgaagctaataccggataacactttttcctacctgggagaaagttaaaagatggcctttgtgctatcactaggggatgggcccgcggcgcattagctagttggtaaggtaatggcttaccaaggcgacgatgcgtagccgacctgagagggtgatcggccacactgggactgagacacggcccagactcctacgggaggcagcagtagggaatcttccgcaatggacgaaagtctgacggagcaatgccgcgtgagtgaggaaggccttcgggtcgtaaagctctgttgtgagggaagaacaagtatcggttgaataagccggtaccttgacggtacctcaccagaaagccacggctaactacgtgccagcagccgcggtaatacgtaggtggcaagcgttgtccggaattattgggcgtaaagcgcgcgcaggcggcttcttaagtctgatgtgaaatctcggggctcaaccccgagcggccattggaaactgggaagcttgagtgcagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccgtagtgccgaagtaaacacattaagcactccgcctggggagtacgaccgcaaggttgaaactcaaaggaattgacggggacccgcacaagcagtggagcatgtggtttaattcgaagcaacgcgaagaaccttaccaggtcttgacatcctttgaccactctggagacagagcttccccttcgggggcaaagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggacgacgtcaaatcatcatgccccttatgacctgggctacacacgtgctacaatggatggtacaaagggttgcgaagccgcgaggtgaagccaatcccataaagccattctcagttcggattgtaggctgcaactcgcctacatgaagccggaattgctagtaatcgcggatcagcatgccgcggtgaatacgttcccgggtcttgtacacaccgcccgtcacaccacgagagtttgtaacacccgaagtcggtgaggtaacctttggagccagccgcctaagg

SEQ ID NO: 7 - DNA, 1417 bp, Alkalihalophilus pseudofirmus
  source 1..1417: mol_type = genomic DNA
tgcaagtcgagcggagtgatgggagcttgctccctgatgttagcggcggacgggtgagtaacacgtgggcaacctgcctgtaagactgggataactccgggaaaccggggctaataccggataacccgttccacctcatggtggagcggtaaaagatggcctctggctatcacttacagatgggcccgcggcgcattagctagttggtaaggtaacggcttaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgtttgaataaggcggcaccttgacggtacctaaccagaaagccacggctaactacgtgccagcagccgcggtaatacgtaggtggcaagcgttgtccggaattattgggcgtaaagcgcgcgcaggcggtctcttaagtctgatgtgaaagcccacggctcaaccgtggagggtcattggaaactgggagacttgagtacagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccttagtgccgaagttaacacattaagcactccgcctggggagtacgaccgcaaggttgaaactcaaaggaattgacgggggcccgcacaagcagtggagcatgtggtttaattcgaagcaacgcgaagaaccttaccaggtcttgacatcctttgaccactctagagatagagctttccccttcgggggacaaagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaaaaccgcgaggttgagcgaatcccataaagccattctcagttcggattgtaggctgcaactcgcctacatgaagccggaattgttagtaatcgcggatcagcatgccgcggtgaatacgttcccgggccttgtacacaccgcccgtcacaccacgagagtttgtaacacccgaagtcggtgaggtaaccttttggagccagc